(19) United States
(12) Patent Application Publication
(10) Pub. No.: US 2020/0178873 A1
(43) Pub. Date: Jun. 11, 2020

(54) MUSCULOSKELETAL ASSESSMENT APPARATUS

(71) Applicants: Matti Palo, Covington, LA (US); Benjamin Legum, Bluemont, VA (US)

(72) Inventors: Matti Palo, Covington, LA (US); Benjamin Legum, Bluemont, VA (US)

(73) Assignee: ORTHO BISTRO LLC, Covington, LA (US)

(21) Appl. No.: 16/211,308

(22) Filed: Dec. 6, 2018

(51) Int. Cl. A61B 5/22; A61B 5/00; A61B 5/107; A61B 5/11; A61B 5/053; A61B 5/01

(52) U.S. Cl. CPC A61B 5/225; A61B 5/0075; A61B 5/0077; A61B 5/1073; A61B 5/1121; A61B 5/1124; A61B 5/7465; A61B 5/0537; A61B 5/015; A61B 5/45; A61B 5/745

(57) ABSTRACT

The present embodiments discloses a musculoskeletal assessment system having a dimensioned base unit including a plurality of sensors configured to transmit medical information corresponding to a patient. Sensors include a motion sensor, a thermographic sensor, a photographic device, and a dynamometer. Each sensor transmits medical information via an integrated computer system.

### TECHNICAL FIELD

The embodiments presented provide an orthopedic and musculoskeletal assessment device configured to measure a variety of collected data corresponding to a patient.

### BACKGROUND

One frequently encountered challenge by medical professionals was to evaluate a patient's physical wellbeing at a targeted area. Current devices require manual operation and are limited to measuring motion and visually inspecting for inconsistencies and abnormalities. These processes are time-consuming, expensive, and inconsistent. Often, once an initial observation and initial diagnosis are made, a referral can be made to a specialist for further analysis and screening. This can result in inconsistencies and disconnect between the patient and their medical data.

The technical resources that go into the delivery of healthcare have been studied extensively. Advances are frequent in the area of diagnostic testing, therapeutics, and pharmaceuticals. While these areas are in constant flux, a need still exists to maximize efficiency within the practice. Since medical professionals only spend a few minutes with each patient, an ever-present need exists to maximize the efficiency, accuracy, and efficacy of this encounter. The ability of the medical specialist to identify the musculoskeletal problem directly impacts the success of any particular treatment protocol.

Current commonly used screening techniques for patients include manual history and physical exam, X-Rays, Mill, and CT scans. These methods have become widely adopted into the practice of treating musculoskeletal problems and addressing joint performance issues. However, each of these methods are limited by subjectivity and transportability which can lead to inaccurate and inconsistent patient data, lengthening the time to recovery. Further, these methods often require multiple specialists and appointments for the problem to be correctly assessed.

Strategies must be developed to enhance the quality of care with the amount of time available. The use of advancing technologies to perform diagnostic screening and evaluations can be utilized to provide the medical specialist with valuable information without requiring the patient to make multiple appointments at various locations. One such advance in orthopedic and musculoskeletal assessment is described in the embodiments presented herein.

### SUMMARY OF THE INVENTION

This summary is provided to introduce a variety of concepts in a simplified form that is further disclosed in the detailed description of the invention. This summary is not intended to identify key or essential inventive concepts of the claimed subject matter, nor is it intended for determining the scope of the claimed subject matter.

Embodiments herein provide a musculoskeletal assessment apparatus having a dimensioned base unit including a support member configured to retain an upper assembly at a first height and a lower assembly at a second height. Each assembly includes a plurality of sensors configured to transmit medical information corresponding to a patient. Sensors can include motion sensors, thermographic sensors and photographic devices, and dynamometers.

It is an aspect of the present invention to capture range of motion data, thermographic data, volumetric data, strength, electrical impedance, and power output, and optical imaging data related to the patient.

In another aspect, a computer system is integrated to receive patient medical data sourced from each sensor disposed within the apparatus. A display can be presented to the patient providing a means of providing instructions and for patient interaction.

In one aspect, each of the upper assembly and lower assembly are comprised of a housing configured to receive an upper surface. Each sensor is dimensioned to be contained within the housing and the upper surface. The upper surface includes apertures configured to receive a transparent cover permitting the passage of sensor input therethrough.

A plurality of fans are configured to cool each of the plurality of sensors and can provide a consistent ambient temperature for the exam. The fans can be configured to provide cooling airflow to the sensors by drawing air into a first aperture and forcing hot air out of a second aperture.

In another aspect of the present invention, the musculoskeletal assessment apparatus provides a multifaceted system for gathering and assessing data related to the target anatomy of the patient. The present embodiments provide a dimensioned apparatus for the analysis of a patient's forearms, elbows, wrists, hands, and fingers as well as lower leg, ankle, foot, and toes by gathering thermographic data, range of motion, volumetric data, strength data, impedance, and visual data related to the target anatomy. Abnormalities can be assessed via a computer system.

Moreover, in accordance with a preferred embodiment of the present invention, other aspects, advantages, and novel features of the present invention will become apparent from the following detailed description in conjunction with the drawings.

### BRIEF DESCRIPTION OF THE DRAWINGS

A more complete understanding of the present invention and the advantages and features thereof will be more readily understood by reference to the following detailed description when considered in conjunction with the accompanying drawings wherein:

FIG. 1 illustrates a perspective view of a patient interacting with the musculoskeletal assessment apparatus, according to some embodiments;

FIG. 2 illustrates a perspective view of the musculoskeletal assessment apparatus, according to some embodiments;

FIG. 3 illustrates an exploded view of the lower assembly, according to some embodiments;

FIG. 4 illustrates a perspective view of the lower assembly and sensors, according to some embodiments;

FIG. 5 illustrates an exploded view of the lower assembly and upper surface, according to some embodiments;

FIG. 6 illustrates a top plan view and a perspective view of the lower assembly, according to some embodiments;

FIG. 7 illustrates a schematic of the musculoskeletal assessment system hardware configuration, according to some embodiments;

FIG. 8 illustrates a perspective view of a patient interacting with the musculoskeletal assessment apparatus, according to some embodiments; and

FIG. 9 illustrates a perspective view of the dynamometer, according to some embodiments.

### DETAILED DESCRIPTION

The specific details of the single embodiment or variety of embodiments described herein are to the described apparatus. Any specific details of the embodiments are used for demonstration purposes only and not unnecessary limitations or inferences are to be understood therefrom.

Any reference to “invention” within this document is a reference to an embodiment of a family of inventions, with no single embodiment including features that are necessarily included in all embodiments, unless otherwise stated. Furthermore, although there may be references to “advantage's” provided by some embodiments, other embodiments may not include those same advantages or may include different advantages. Any advantages described herein are not to be construed as limiting to any of the claims.

Before describing in detail exemplary embodiments, it is noted that the embodiments reside primarily in combinations of components related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second” and the like, may be used solely to distinguish one entity or element from another entity or element without necessarily requiring or implying any physical or logical relationship or order between such entities or elements.

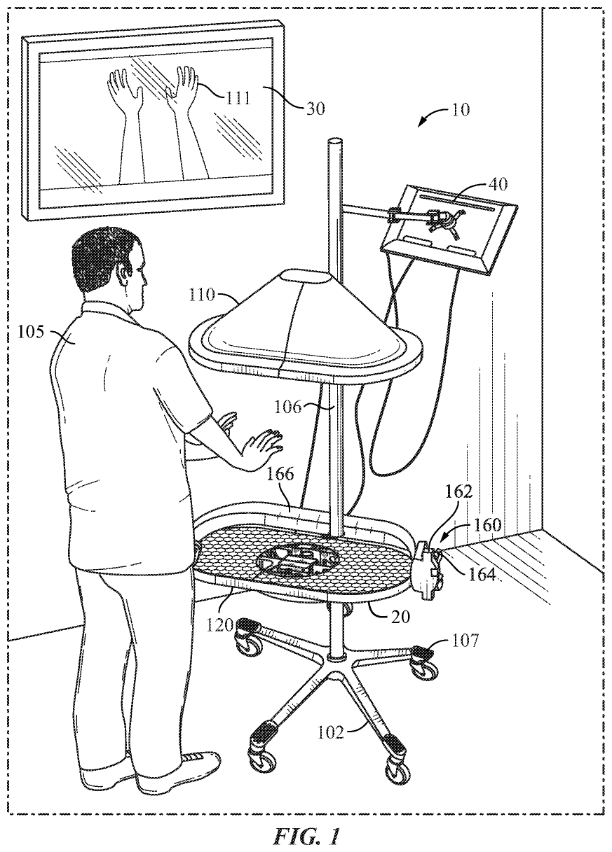

The embodiments presented herein provide an orthopedic and musculoskeletal assessment apparatus 10 having a variety of sensors disposed within a base unit 20 dimensioned to fit around the patient's 105 hands and wrist, as shown in FIG. 1. The interior of the base unit 20 includes a plurality of sensors which may move throughout the base unit to collect data corresponding to a selected area of the patient's body and the movement thereof, volume, electrical resistance, temperature, power output (i.e., strength), and aesthetic appearance. The apparatus 10 may be independent with respect to the patient 105 and does not require any fastening, mounting, or retainment on the body of the patient 105 providing added comfort during the encounter.

While the present embodiment provides for a base unit 20 configured to scan the hand, fingers, wrist, and forearm and elbow of the user, the base unit can be configured in a variety of dimensions such that alternate areas and specific joints can be scanned. In some embodiments, the device can be reconfigured to scan the foot and ankle of the patient such that the base is pivoted to scan the side of the patient.

A variety of components comprise the apparatus 10. It is understood that fastening, adhesion, coupling, and joining of a component or set of components with one another can be done by mechanical, chemical (adhesives), molding, or similar means of attachment. Attachment means can be presented in combination with one another, such as applying an adhesive before mechanically fastening components with others. Various forms of screws, fasteners, bolts, adhesives, and similar coupling means can be implemented without departing from the spirit of the embodiments presented.

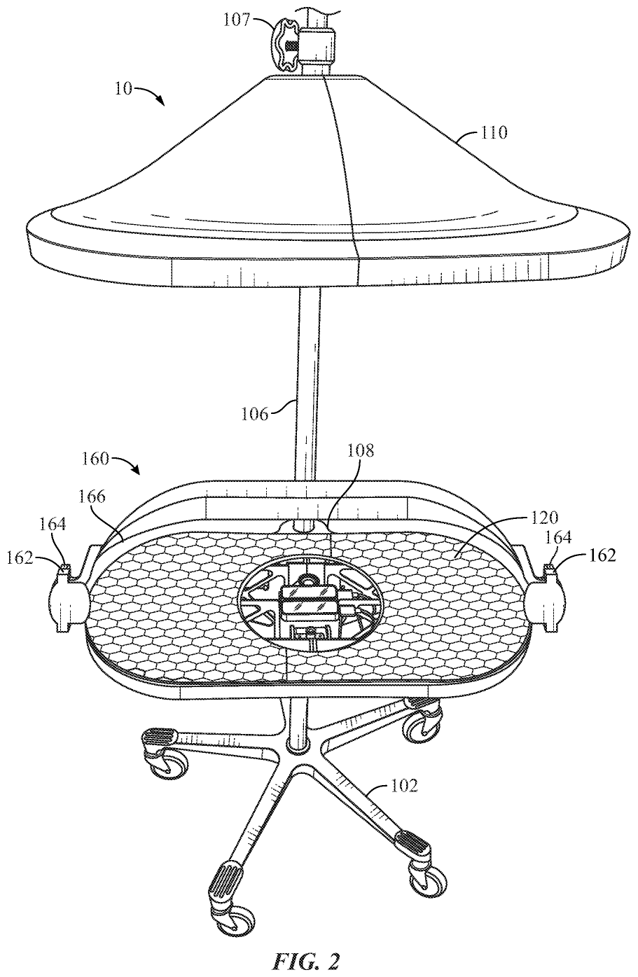

In reference to FIG. 1 and FIG. 2, the apparatus 10 is illustrated comprising an upper assembly 110 and a lower assembly 120. Each of the upper and lower assemblies 110,120 are constructed, assembled, and function uniformly. For simplicity, the lower assembly 120 is discussed in detail throughout with the understanding that the upper assembly 110 is positioned to vertically mirror the lower assembly 120 as shown in FIGS. 1 and 2. An aspect of the invention is directed to an apparatus 10 mounted to a support frame base 100 constructed to support the upper assembly 110 and lower assembly 120. The base assembly 102 rests movably on the floor or similar surface. In at least one embodiment, the base assembly 102 includes a plurality of wheels allowing the apparatus 10 to be quickly moved. Each wheel can include a locking mechanism 107 to ensure the stability of the apparatus 10 during an interaction with the patient 105.

The lower assembly 110 is positioned at a first height, and the upper assembly 120 is positioned at a second height along the length of the member 106. In one aspect, the height of first and second heights are adjustable dependent on patient 105 height or anatomy of interest. In the illustrated embodiment, member 106 is configured as an elongated cylindrical member to support the weight of the lower and upper assemblies 120, 110. Each of the upper and lower assemblies 110, 120 are movably coupled to the member 106 via pivoting members 108 configured as an aperture to permit the member 106 to pass therethrough. Pivoting member 108 can permit each of the lower assembly and upper assembly 110, 120 to optionally rotate around any axis relative to the support frame base 100. Preferentially, each assembly 110, 120 rotates around the member 106. Rotation can be optionally powered by an actuator placed between the member 106 and each assembly 110, 120. Alternate embodiments permit manual rotation of each assembly 110, 120 which can each be locked into the desired position.

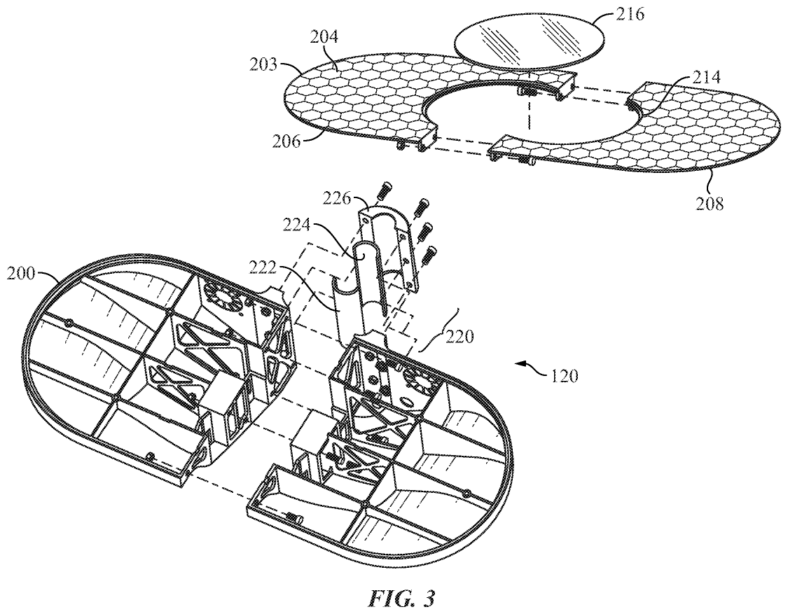

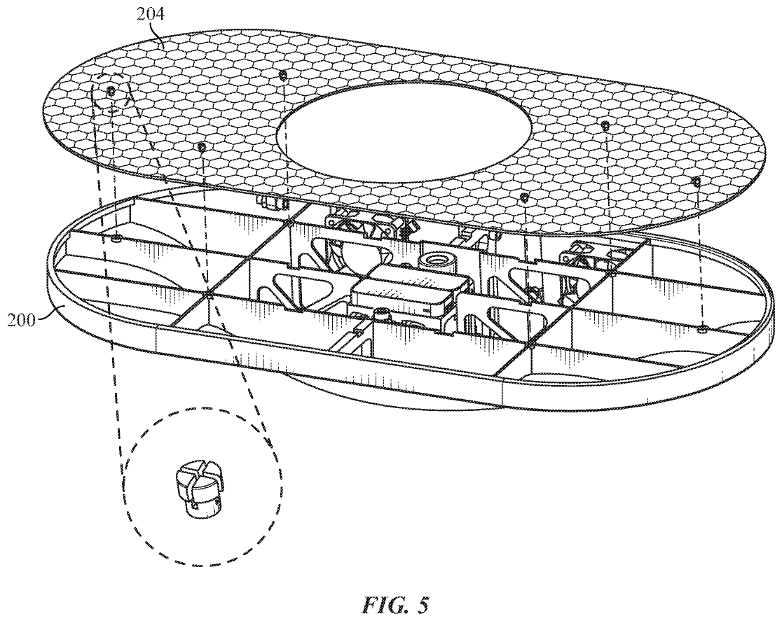

FIG. 3 illustrates an exploded view of the lower assembly 110 comprised of a housing 200 dimensioned to contain a plurality of sensors. The lower assembly 110 includes a releasably engaged upper surface 204 to fasten to the housing 200. In one embodiment, the housing edge 201 and upper surface edge 203 are similarly dimensioned such that one either component is received by the other (see FIG. 5). The upper surface 204 is configured having a left side 206 and right side 208 which are fastened to one another at connection points at a medial axis. The aperture 214 is dimensioned to receive a transparent, or partially transparent cover 216 which permits light to pass therethrough to the sensors within the lower assembly 110. The transparent cover 216 can be constructed of a suitable transparent or semi-transparent material including polymethyl methacrylate (“PMMA”) or similar thermoplastic utilized when tensile strength, flexural strength, transparency, polishability, and UV tolerance are more important than impact strength, chemical resistance, and heat resistance.

A mounting assembly 220 can fasten to the proximal side 221 (in relation to the member 106). The mounting assembly can include inner sleeve components 222, 224 to fittingly engage the member 106. A mounting assembly housing 226 retains the inner sleeve components 222, 224 therein. Each sleeve 222,224 provides a friction fit to member 106 retaining each of the upper assembly 110 and lower assembly 120 at a first and second height respectively.

The housing 200 can be constructed as a truss having cross members spanning both the length and width of the housing 200 to intersect one another. The truss cross members provide an anchor point for each sensor or another interior component in the lower assembly 120.

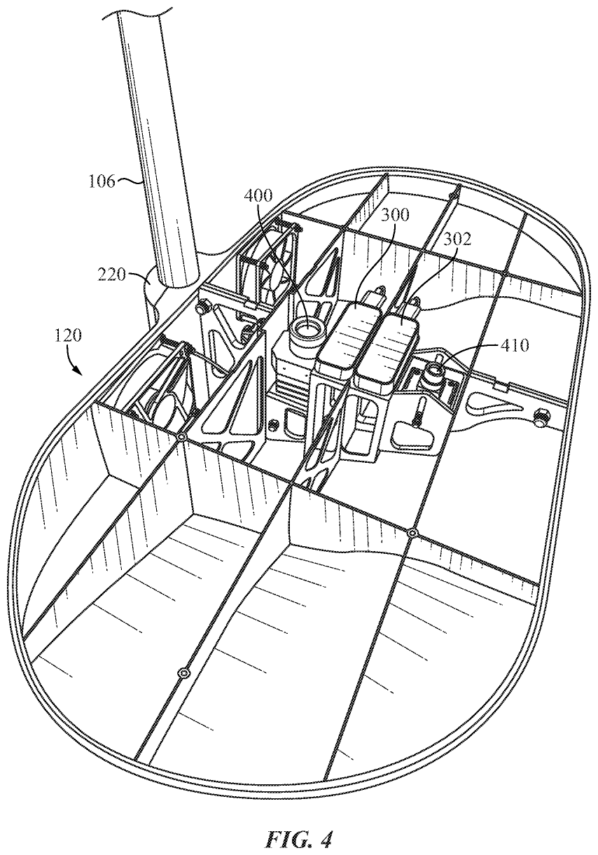

As mentioned, the apparatus 10 includes a plurality of sensors as illustrated in FIGS. 4-6. Each sensor is configured to gather data in relation to the patient 105 and output the data for analysis. In one aspect, the apparatus 10 includes at least one sensor configured as two or more motion sensors 300, 302. During use, a patient places his or her hands within the range of the motion sensor(s) 300, 302 and move their arm, wrist, hand, or finger. The system may be provided with a virtual visual stimulus to allow the patient to interact with a virtual routine, thus providing an impetus for movement. In one embodiment, the motion sensors 300, 302 include cameras and infrared Light Emitting Diodes (LED's). Each LED is used to track infrared light outside the visible light spectrum. Each camera has a wide-angle lens to permit a broad interaction space.

The invention may or may not require the patient to interact with virtual objects. However, the use thereof is contemplated. In any interaction, data is gathered related to the patient's 105 motion and transmitted to a computer system for further processing. The infrared sensor 300,302 is utilized to track the patient's 105 hands as they perform a range-of-motion routine. It is also used to measure the joint and body part angles which are ultimately used to calculate the motion of the patient's hands and wrists. This motion scanning system 300, 302, and the infrared camera motion sensor of the present embodiment can be utilized to calculate volumetric measurements of the hands and fingers.

In a preferred embodiment, a pair of motion sensors 300, 302 adhere to a surface of the housing 200 within each of the upper and lower assemblies 110, 120. Each motion sensor 300, 302 is positioned such that the cameras and infrared LED's can collect and emit light through the transparent cover 216 to collect data from the patient. A first motion sensor 300 can be configured to track the motion of the patient 105, while the second motion sensor 302 is configured for interaction with a virtual object.

In one embodiment, the system includes a pair of light sources which can be disposed to either side of the motion sensors 300. Light sources can be infrared light sources of generally conventional design, e.g., infrared light-emitting diodes (LEDs), and cameras can be sensitive to infrared light. A set of filters can be placed in front of the cameras to filter out visible light so that only infrared light is registered in the images captured by cameras. In some embodiments where the object of interest is a person's hand or body, use of infrared light can allow the motion-capture system to operate under a broad range of lighting conditions and can avoid various inconveniences or distractions that may be associated with directing visible light into the region where the person is moving. However, a particular wavelength or region of the electromagnetic spectrum is required.

For example, lasers or other light sources can be used instead of LEDs. For laser setups, additional optics (e.g., a lens or diffuser) may be employed to widen the laser beam (and make its field of view similar to that of the cameras). Useful arrangements can also include short and wide-angle illuminators for different ranges. Light sources are typically diffuse rather than specular point sources; for example, packaged LEDs with light-spreading encapsulation are suitable.

In a preferred embodiment and in further reference to FIG. 4 the apparatus includes image sensors 400 and 410. A variety of imagery can be captured, however, in the present embodiment, a thermal imaging device and high definition photography device is used to capture data related to the patient. Thermographic data is captured using either infrared imaging or thermal imaging devices known in the arts. This device can be fastened within both the upper and lower assemblies 110, 120. This data can be used to assess swelling and other joint-related concerns. Photography is used to capture images of the patient. In a preferred embodiment, each camera is positioned on either side of the motion sensors 300, 302.

In alternate embodiments, each camera can be any type of camera, including visible-light cameras, infrared (IR) cameras, ultraviolet cameras or any other devices (or combination of devices) that are capable of capturing an image of an object and representing that image in the form of digital data. The cameras are preferably capable of capturing video images (i.e., successive image frames at a constant rate of at least 15 frames per second), although no particular frame rate is required. The particular capabilities of the cameras are not critical to the invention, and the cameras can vary as to frame rate, image resolution (e.g., pixels per image), color or intensity resolution (e.g., number of bits of intensity data per pixel), focal length of lenses, depth of field, etc. In general, for a particular application, any cameras capable of focusing on objects within a spatial volume of interest can be used. For instance, to capture the motion of the hand of an otherwise stationary person, the volume of interest might be a meter on a side.

In the preferred embodiment, the strength of the patient is assessed using a dynamometer 160 having a handle 162, a grip 164, and a support member 166. In the present embodiment, the dynamometer 160 is configured to assess hand, pinch and grip strength. In one example, bilateral hand dynamometers are utilized. Each dynamometer can be incorporated into the apparatus or presented as separate devices in communication with the apparatus and analysis system. The impedance sensor may be incorporated into the handle of the dynamometer.

In some embodiments, the apparatus includes an impendence sensor configured to measure body composition of the patient.

Some embodiments of the invention can comprise linear displacement sensors that are utilized on hinge bars to determine what length setting the patient has selected. In some embodiments, positional sensors can be used to determine which ROM stops have been engaged, and compared to what ROM limits should or should not be employed. The apparatus described herein can comprise force sensors, torque sensors, and a dynamometer that can be integrated to determine the strength or force/torque output of the joint and correlated to the recovery of the patient.

In some further embodiments, at least one of the assemblies described herein can comprise an electromyography sensor, a strain gage sensor or other sensor configured to measure strains continuously or intermittently. In some embodiments, these measurements can be used to assess motion, deflection, or provide quantifiable data of muscle growth, muscle contraction, or forces, torques or pressures resulting from a muscle contraction. The muscle contraction may be voluntary or involuntarily elicited via electrical muscle stimulation. In some embodiments, the data collected from the electromyography sensor or strain gage sensor can be utilized in a closed loop feedback control methodology to optimize/customize the electrical stimulation parameters to provide the most efficient or strongest muscle contraction for the patient. The data can also be utilized by the healthcare provider to fine tune the treatment programs based on the patient's data captured from the electromyography or strain gage sensor.

Furthermore, the apparatus can be adapted and configured to engage a medical diagnostic device configured to capture data on the subject. Medical diagnostic devices typically include, for example, any device having a sensor adapted and configured to capture data from the subject (patient). For example, X-ray scanners, X-ray tubes with image intensifier tube, magnetic resonance scanners, infrared cameras, computed tomography scanners, ultrasound scanners, electromyography sensor units, digital camera and cameras, and electromyography sensor unit with sensors attached to the subject. The apparatus can be adapted and configured such that the medical diagnostic device detachably connects to the apparatus.

Alternative embodiments can be configured to perform pulse oximetry and can comprise a non-invasive blood pressure sensor configured to measure arterial blood pressure continuously or intermittently. In some further embodiments, a patient's heart-rate can be measured in addition to sensing the patient's blood pressure. In some embodiments, one or more of the brace systems or assemblies described herein can include at least one blood pressure sensor integrated with a portion the apparatus. In other embodiments, the apparatus can include at least one blood pressure sensor coupled to an adjacent to or some distance from the apparatus. In other embodiments a transcutaneous blood sugar monitor may be employed.

Each sensor is dimensioned to be within the housing 200 and upper surface 204 as shown in FIG. 5.

The present invention contemplates compatibility with all types of diagnostic imaging that are capable of producing moving images of joint motion. The method typically utilizes videofluoroscopy technology, CT scans, and magnetic resonance imaging. However, other diagnostic imaging methods such as ultrasound imaging, and imaging methods. In addition, three-dimensional imaging platforms could be employed if the motion control devices could move along a three-dimensional surface, as opposed to within a two-dimensional plane, as is contemplated in the preferred embodiment of the motion control device. One skilled in the art will appreciate that as additional medical scanning or diagnostic devices become available, the present invention can be adapted to accommodate them.

The present invention contemplates the use of surface electromyography for the measurement of muscle involvement, however other diagnostic systems may be used as well in an alternative embodiment such as Mill and ultrasound or other technologies. These other diagnostic systems may or may not be sensor based. One skilled in the art will appreciate that as additional medical scanning or diagnostic devices become available, the present invention can be adapted to accommodate them.

Other interpretation methodologies other than those listed: methodologies for interpreting the measurements are provided by the present invention to generate diagnostic results that can be clinically applied in the treatment of subjects with joint problems or performance issues. While these aspects are necessary for reducing to practice the diagnostic apparatus and methods given in the first, second, and third aspects of the invention, these aspects may be alternatively embodied by other interpretation methodologies that can be applied to the diagnostic measurements afforded by the apparatus and method of the present embodiments. These might include applications of the diagnostic measurements outside of the boundaries of validation that are provided for through controlled clinical trials using the diagnostic apparatus and method. These might also include less structured interpretation methodologies, and methodologies applied by practitioners other than therapists, physicians, surgeons, chiropractors, veterinarians, and other health professionals.

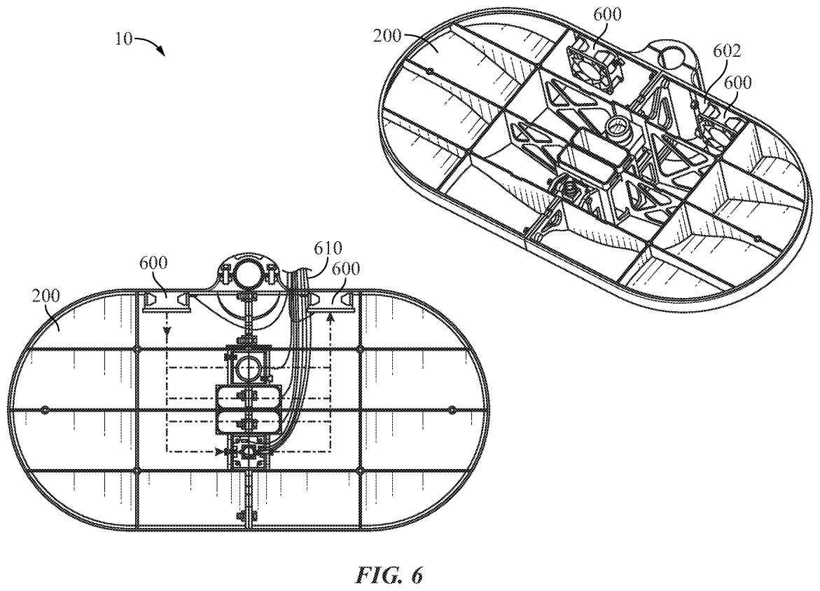

In reference to FIG. 6, a plurality of fans 600 are configured to force air from the interior of the housing 200 to the exterior to actively cool each of the lower and upper assemblies 110, 120. In one aspect, each fan 600 is positioned on the interior sidewall 602 of the housing 200 in fluid communication with an exhaust aperture providing a means for hot air to flow out of each assembly 110,120. In one embodiment, at least one of the plurality of fans 600 is configured to draw air into the apparatus along air flow travel path T₁ while a second fan forces air to the exterior along travel path T₂ creating a cooling flow of air through the interior of the housing 200 to actively cool each sensor with air from the exterior.

The component wiring 610 provides means for an electrical connection between each sensor and computer components of the apparatus 10.

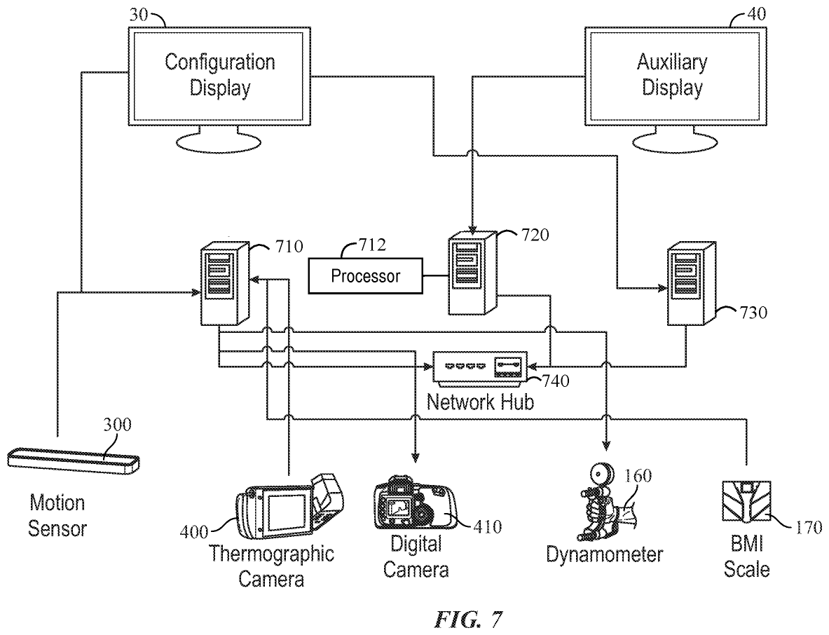

In reference to FIG. 7, each sensor is in communication with a computer system. At least one display is presented to the user. The display can provide instructions to the user as well as means for interacting with a virtual object or environment. Auxiliary displays showing data can be used by medical professionals. It will be appreciated that the computer system is illustrative and that variations and modifications are possible. Computers can be implemented in a variety of form factors, including server systems, desktop systems, laptop systems, tablets, smartphones or personal digital assistants, and so on. A particular implementation may include other functionality not described herein, e.g., wired and/or wireless network interfaces, media playing and/or recording capability, etc. In some embodiments, one or more cameras may be built into the computer rather than being supplied as separate components.

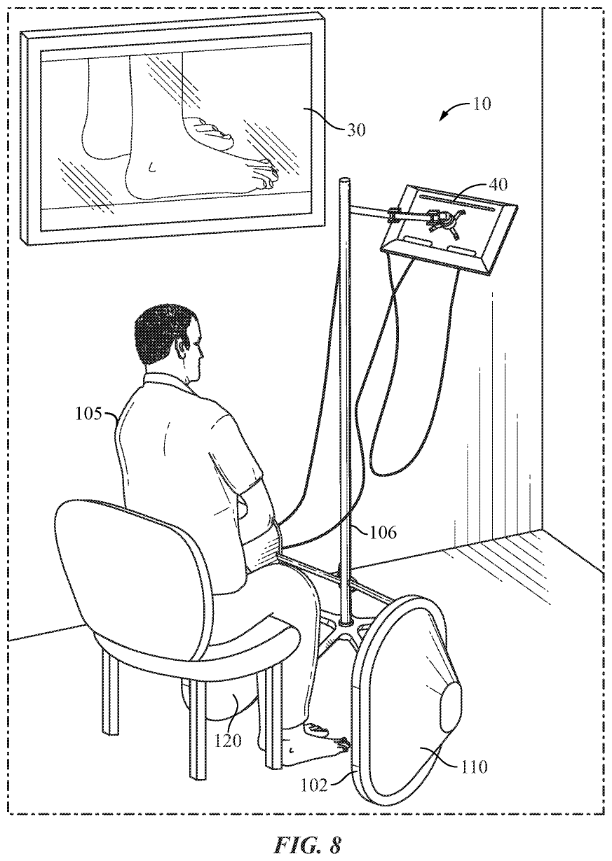

FIG. 8 illustrates an embodiment wherein the upper assembly 110 and lower assembly 120 are pivoted to permit the patient 105 to stand therebetween. With the illustrated embodiment, the apparatus can now scan the knee, lower leg, ankle, foot, and toes of the patient 105.

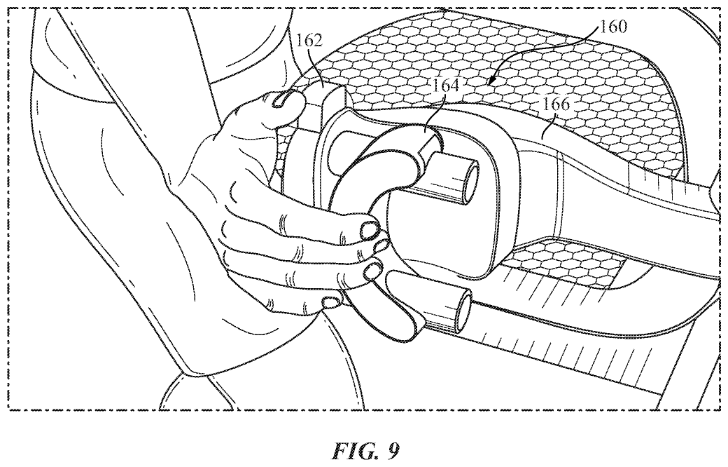

FIG. 9 illustrates a detailed view of the dynamometer 160 assembly. Bilateral supports 166 may be positioned at each side of the lower assembly 120, or suitably positioned elsewhere on the apparatus to permit the engagement with the patient's hands to assess grip and pinch strength. In some embodiments, the dynamometer includes a grip 162 and handle 164 having various sensors measuring interactions with the patient.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subconibinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A musculoskeletal assessment apparatus comprising:
a dimensioned base unit including a plurality of sensors configured to transmit medical information corresponding to a patient.

2. The apparatus of claim 1, further comprising a support member configured to retain an upper assembly at a first height and a lower assembly at a second height.

3. The apparatus of claim 2, wherein each of the upper assembly and lower assembly includes the plurality of sensors, wherein the plurality of sensors includes at least one of the following:
at least one infrared sensor;
at least one impedance sensor;
at least one photographic device; and
at least one dynamometer.

4. The apparatus of claim 1, wherein the plurality of sensors are configured to calculate at least;
volumetric data;
range of motion data electrical resistance;
thermographic data;
pinch, grip and hand strength; and
optical imaging data.

5. The apparatus of claim 1, wherein each of the upper assembly and lower assembly is comprised of a housing configured to receive an upper surface, wherein each of the plurality of sensors is dimensioned to be contained within the housing and the upper surface.

6. The apparatus of claim 5, wherein the upper surface includes an aperture configured to receive a transparent cover, wherein the transparent cover permits passage of sensor input therethrough.

7. The apparatus of claim 1, further comprising a plurality of fans configured to cool each of the plurality of sensors and provide a consistent ambient temperature.

8. The apparatus of claim 1, the base assembly is dimensioned to engage with the patient's hand, forearms, foot, and ankle.

9. The apparatus of claim 1, further comprising a computer system configured to receive patient data from the plurality of sensors.

10. The apparatus of claim 1, further comprising a display to provide an interactive experience to the patient.

11. A musculoskeletal assessment apparatus comprising:
a dimensioned base unit including a support member configured to retain an upper assembly at a first height and a lower assembly at a second height, each of the upper assembly and lower assembly including a plurality of sensors configured to transmit medical information corresponding to a patient, wherein the plurality of sensors includes at least one of the following:
at least one infrared sensor;
at least one impedance sensor;
at least one photographic device; and
at least one dynamometer.

12. The apparatus of claim 11, wherein the plurality of sensors are configured to calculate at least;
volumetric data;
range of motion data electrical impedance thermographic data;
pinch, grip and hand strength; and
optical imaging data.

13. The apparatus of claim 11, wherein the motion sensor includes at least one camera and at least one infrared Light Emitting Diode.

14. The apparatus of claim 11, further comprising a computer system configured to receive patient data from the plurality of sensors.

15. The apparatus of claim 11, further comprising a display to provide an interactive experience to the patient.

16. The apparatus of claim 11, wherein each of the upper assembly and lower assembly are comprised of a housing configured to receive an upper surface, wherein each of the plurality of sensors is dimensioned to be contained within the housing and the upper surface.

17. The apparatus of claim 16, wherein the upper surface includes an aperture configured to receive a transparent cover, wherein the transparent cover permits passage of sensor input therethrough.

18. The apparatus of claim 11, further comprising a plurality of fans configured to cool each of the plurality of sensors.

19. A musculoskeletal assessment apparatus, the apparatus comprising:
a base unit including a support member to secure an upper and lower assembly to house a plurality of electronically connected sensors configured to transmit a plurality of medical information corresponding to a predetermined patient;
a plurality of sensors configured along the upper and lower assembly including:
at least one infrared sensor;
at least one impedance sensor
at least one dynamometer; and
at least one camera.

20. The apparatus of claim 19, wherein the plurality of medical information corresponding to the pre-determined patient include:
a volumetric data set;
a body composition set;
a thermographic data set;
an optical imaging data set; and
data corresponding to a grip and hand strength.
